(19) United States
(12) Patent Application Publication
(10) Pub. No.: US 2024/0395023 A1
(43) Pub. Date: Nov. 28, 2024

(54) A COMPUTER-IMPLEMENTED METHOD, DATA PROCESSING APPARATUS, AND COMPUTER PROGRAM FOR ACTIVE LEARNING FOR COMPUTER VISION IN DIGITAL IMAGES

(71) Applicant: UCL BUSINESS LTD., London (GB)

(72) Inventors: Watjana LILAONITKUL, London (GB); Adam DUBIS, London (GB); Mustafa ARIKAN, London (GB)

(73) Assignee: UCL BUSINESS LTD., London (GB)

(21) Appl. No.: 18/694,711

(22) PCT Filed: Sep. 22, 2022
(86) PCT No.: PCT/GB2022/052404

§ 371 Date: Mar. 22, 2024

(30) Foreign Application Priority Data
Sep. 23, 2021 (GB) 2113613.0

(51) Int. Cl. G06V 10/774; G06V 10/762; G06V 10/764; G06V 10/776; G06V 10/82; G16H 30/40

(52) U.S. Cl. CPC G06V 10/774; G06V 10/762; G06V 10/764; G06V 10/776; G06V 10/82; G16H 30/40; G06V 2201/03

(57) ABSTRACT

A computer-implemented method of active learning for computer vision in digital images, comprising: inputting labelled image training examples into an artificial neural network in a training phase; training a computer vision model using the labelled training examples; carrying out a prediction task on each image of an unlabelled training set of unlabelled, unseen images using the model; calculating an uncertainty metric for the predictions in each image of the unlabelled training set; calculating a similarity metric for the unlabelled training set representing similarities between the images in the training set; selecting images from the unlabelled training set, in dependence upon both the similarity metric and the uncertainty metric of each image, to design a training set for labelling which tends to both lower the similarity between the selected images and increase the uncertainty of the selected images.

### FIELD OF THE INVENTION

The present invention relates to methods of active-learning in the field of computer vision.

### BACKGROUND OF THE INVENTION

Two important aspects in the development of deep learning technologies as part of a decision support system are data efficiency, and performance robustness. In many imaging applications, deep learning (DL) has been shown to achieve excellent results, but these algorithms require large amounts of labelled data. However, in many fields of research such as medicine, the acquisition process for high-quality labelled data can be prohibitively tedious and time-consuming.

One strategy to overcome this limitation is to increase data efficiency by employing an active learning protocol during the training phase. Active learning (Cohn et al., 1996) entails a system learning from data, and choosing automatically what additional data it needs for experts to label in order to efficiently improve system performance. In active learning (Cohn et al., 1996), an acquisition function is used to score and prioritise new training samples to add to an initial training set in order to increase the performance gains per unit training sample. As experts only need to label the selected samples, active learning can make the task of achieving a certain level of accuracy easier and more cost-effective.

Known active learning processes commonly use acquisition functions that select samples based on model uncertainty. To alleviate the burden of manual annotation and minimize the cost, active learning has been proposed (Yang et al., 2017) for biomedical segmentation. However, they rely on training multiple models to estimate uncertainty, which slows down development. Uncertainty estimation using Monte Carlo (MC) dropout (Gal and Ghahramani, 2016; Kwon et al., 2019) allows estimation of both epistemic and aleatoric uncertainty using a single model. Using dropout at test-time has been shown to improve performance and make annotation cost-effective (Górriz et al., 2017). In case of 3D medical volumes active learning has been proposed to select informative 2D slices for a sparse annotation strategy in medical image segmentation (Zhang et al., 2019). A method for active and incremental fine-tuning has been proposed (Zhou et al., 2017) to integrate active learning and transfer learning. Active learning in combination with uncertainty estimation has also been proposed for a region-based query method (Li and Alstrøm, 2020) for medical image segmentation using methods such as VarRatio, Entropy and BALD (Gal et al., 2017; Beluch et al., 2018).

However, the inventors have come to the realisation that active learning based solely on a type of uncertainty measure can become susceptible to selection inefficiencies when the selected data with the highest associated uncertainty measure happens to cluster within a small subset of classes. The unintended consequence is a class imbalance that may negatively impact both the prediction performance and the prediction robustness as the features learned by the DL network become more skewed to those within the majority class in the selected training samples.

What is a desired is a better active learning process that addresses the limitation of known methods and significantly improves data efficiency, prediction performance, and performance robustness.

### SUMMARY OF THE INVENTION

According to an aspect of the invention, there is provided a computer-implemented method of active learning for computer vision tasks (e.g., segmentation) in digital images, comprising: inputting labelled image training examples into an artificial neural network in a training phase; training a computer vision model using the labelled training examples; carrying out a prediction task on each image of an unlabelled training set of unlabelled, unseen (not previously used in the neural network) images using the model; calculating an uncertainty metric for the predictions in each image of the unlabelled training set; calculating a similarity metric for the unlabelled training set representing similarities between the images in the unlabelled training set; selecting images from the unlabelled training set, in dependence upon both the similarity metric and the uncertainty metrics of each image, to design a (reduced) training set for labelling which tends to both lower the similarity between the (potential) selected images and increase the uncertainty of the selected images.

The inventors have provided a method that may be used with Bayesian DL, for example in segmentation. They have come to the realisation that it is possible to use metrics for labelling a select number of examples from the unlabelled pool of images which are both dissimilar and have a high level of uncertainty in their prediction output by the (computer vision) neural network model. Ranking (and selection) using uncertainty estimation alone may result in similar looking images, particularly, as an example, for scans in 3D medical volumes. Hence the inventors have designed a method in which images in a training set are selected for labelling based on both uncertainty and similarity of the images to each other. Similarity or uncertainty may be considered in either order or simultaneously to select the images, as long as the selected images are both a diverse selection (of features in the images) and each has a high level of uncertainty of prediction.

The percentage selection (for the labelling of the training set) may be any suitable percentage which is less than 100% of the images, and preferably less than 20% of the images, such as 5, 10, or 15%. It is sometimes preferable to have a relatively large testing set (for instance, 60% or higher of the images) to ensure accurate calculation of model performance. A large testing set can help for comparing strategies, because while some strategies may perform worse with additional examples others can be robust.

The method may include further stages. For example, the method may further comprise: outputting the training set for labelling to an expert (such as a human expert); inputting the same images of the training set for labelling further including labels added by the expert as a labelled training set into the artificial neural network; further training the model using the labelled training set; carrying out the prediction task on new images in an inference phase using the refined segmentation model.

These further stages use the training carried out using the additional training examples created by the labelled training set as set out above.

Any suitable uncertainty metric may be used. In some examples, the uncertainty metric is based on a Monte Carlo, MC, dropout method of estimating uncertainty.

The uncertainty metric may be based on aleatoric or epistemic uncertainty, or both. Hence, any combination of aleatoric and epistemic uncertainty may be provided by the uncertainty metric. Depending on the dataset, better results may be obtained using one or the other type of uncertainty in addition to similarity, or both.

For instance, the uncertainty metric may be a global uncertainty metric including both types of uncertainty and thus incorporating estimation of both an epistemic uncertainty and an aleatoric uncertainty metric. The global uncertainty metric (or any uncertainty metric) may be estimated and used for ranking of images (before or after similarity is considered).

The uncertainty metric may use Bayes' theorem to find a posterior distribution over convolutional weights W, given observed training data X and labels Y.

As one example, the prediction task may be segmentation, and the computer vision model may be a segmentation model (for classification of individual pixels). In this case, the segmentation model records (predicted) classification data of the pixels in the image. Hence, uncertainty may be uncertainty of pixel classification.

The uncertainty values for different pixels and classes may be estimated and summed into a single scalar value for ranking of images.

Turning now to similarity, the similarity algorithm may group the images by clustering of similar images. Suitable clustering methodologies (for use on unlabelled data) are known to the person skilled in the art.

In one example, the similarity algorithm clusters similar images into a single group, to provide a number of groups K each containing similar images and calculates a structural similarity index in matrix form of N×N entries giving similarity between every image in the unlabelled training set. The N×N entries may be used to find the K clusters.

Clustering into groups of similar images may take place before or after uncertainty is considered. In the case in which similarity is treated first by grouping, the image with the highest uncertainty level of the uncertainty metric in each group may be selected for labelling from the training set. Of course, more than one image may be selected, for example, assuming the images are ranked for uncertainty within the group, the top-level X images may be selected, where X may be 1 or more than 1.

There may be further active learning iterations including new images from the unlabelled training set. In any subsequent active learning iteration, further selection may take the highest uncertainty level image(s) from the remaining unselected images in each group.

The method may further comprise extending the labelled training examples by adding perturbed images of the labelled training examples, for example including Gaussian noise and/or gamma adjustment to change contrast and brightness.

Any image may be used with the above-defined methods. The methods are well-suited to medical images, and maybe be taken from volumetric data or videos, preferably provided by scanners or cameras.

Embodiments of another aspect include a data processing apparatus, which comprises means suitable for carrying out a method of an embodiment.

Embodiments of another aspect include a computer program comprising instructions, which, when the program is executed by a computer, cause the computer to carry out the method of an embodiment. The computer program may be stored on a computer-readable medium. The computer-readable medium may be non-transitory.

Hence embodiments of another aspect include a non-transitory computer-readable medium comprising instructions, which, when the program is executed by a computer, cause the computer to carry out the method of an embodiment.

The invention may be implemented in digital electronic circuitry, or in computer hardware, firmware, software, or in combinations thereof. The invention may be implemented as a computer program or a computer program product, i.e., a computer program tangibly embodied in a non-transitory information carrier, e.g., in a machine-readable storage device or in a propagated signal, for execution by, or to control the operation of, one or more hardware modules. A computer program may be in the form of a stand-alone program, a computer program portion, or more than one computer program, and may be written in any form of programming language, including compiled or interpreted languages, and it may be deployed in any form, including as a stand-alone program or as a module, component, subroutine, or other unit suitable for use in a data processing environment.

The invention is described in terms of particular embodiments. Other embodiments are within the scope of the following claims. For example, the steps of the invention may be performed in a different order and still achieve desirable results.

### BRIEF DESCRIPTION OF THE DRAWINGS

Reference is made, by way of example only, to the accompanying drawings in which:

FIG. 1 is a flow chart of a method for active learning for segmentation in digital images according to embodiments;

FIG. 2 is a schematic diagram of the training strategy according to embodiments;

FIG. 3a is an example OCT scan;

FIG. 3b is a prediction result of the example OCT scan in FIG. 3a, according to an embodiment;

FIG. 3c is an uncertainty estimation map of the prediction result in FIG. 3b, according to an embodiment;

FIG. 4a is a comparative diagram of test accuracy using an IoU metric for average retinal layer segmentation for select strategies according to an embodiment;

FIG. 4b is a comparative diagram of test accuracy using an IoU metric for average retinal layer segmentation for all strategies according to an embodiment;

FIGS. 5a to 5f are comparative diagrams of test accuracy using an IoU metric for individual retinal layer segmentation for select strategies according to an embodiment;

FIG. 6 is a table showing the Area Under the Learning Curve (AULC) for average and individual retinal layer segmentation for all strategies according to an embodiment;

FIG. 7 is a comparative diagram of drop of IoU test accuracy according to an embodiment;

FIG. 8a is a comparative diagram of uncertainty estimate values for epistemic uncertainty according to an embodiment;

FIG. 8b is a comparative diagram of uncertainty estimate values for aleatoric uncertainty according to an embodiment;

FIGS. 9a and 9b are comparative diagrams of mean IoU score for select training procedures for retinal layer segmentation for a different pathology according to an embodiment;

FIGS. 10a and 10b are comparative diagram of mean IoU score for models fine-tuned for a different pathology and applied to original pathologies, for select strategies;

FIG. 11 is a comparative diagram of test accuracy using an IoU metric for average video image segmentation for select strategies according to an embodiment;

FIG. 12 is a table showing the AULC for average and individual class video image segmentation for select strategies according to an embodiment;

FIGS. 13a to 13g are comparative diagrams of test accuracy using an IoU metric for individual class video image segmentation for select strategies according to an embodiment;

FIG. 14a is an example retinal OCT scan slice;

FIG. 14b is a retinal layer segmentation of the example retinal OCT scan slice;

FIG. 15 is a set of three retinal OCT scan slice pairs, before (top row) and after (bottom row) pre-processing for flattening and alignment;

FIG. 16 is a comparative diagram of test accuracy using an IoU metric for clustering, no clustering, and random selection strategies, where test images are subject to adversarial perturbations;

FIG. 17 is a comparative diagram of test accuracy using an IoU metric for clustering, no clustering, and random selection strategies, where test images are subject to a domain shift;

FIG. 18 is a table showing the AULC for clustering, no clustering, and random selection strategies, where test images are subject to adversarial perturbations and test images are subject to a domain shift;

FIG. 19 is a groups of images showing prediction results in the event of a domain shift, where training images are acquired using a different imaging system to testing images;

FIG. 20 is a comparative diagram of test accuracy using an IoU metric for clustering, no clustering, and random selection strategies, where test images are demonstrative of a different underlying disease relative to the training dataset;

FIG. 21 is a table showing the AULC for clustering, no clustering, and random selection strategies, where test images are demonstrative of a different underlying disease relative to the training dataset;

FIG. 22 is a groups of images showing prediction results in the event of a domain shift, where test images are demonstrative of a different underlying disease relative to the training dataset;

FIG. 23 is a diagram of suitable hardware for implementation of invention embodiments.

### DETAILED DESCRIPTION

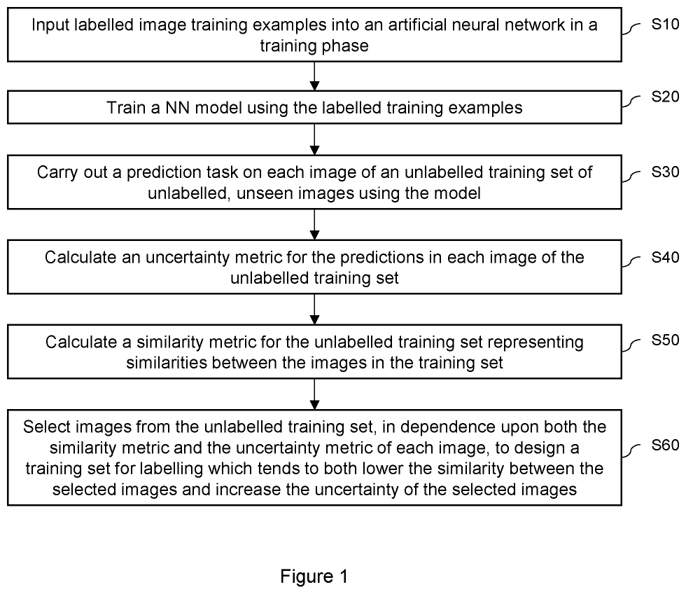

FIG. 1 is a flow chart depicting a computer-implemented method of active learning for segmentation in digital images according to aspects of embodiments of the present invention.

At S10, methods input labelled image training examples into an artificial neural network (such a DL network). These input labelled training example images are used for the training phase of the initial iteration(s) of the network.

At S20, methods train the artificial neural network in order to train a neural network model. Many different computer vision models may be trained during this process, for instance semantic or instance segmentation models, feature extraction, or object detection. Training occurs using the labelled training examples.

At S30, methods validate the trained computer vision model on unlabelled, previously unseen (by the training model) images by performing a prediction task (the target of the task differing on the exact computer vision model in consideration). For example, in image segmentation, the computer vision model may segment or partition the images to locate objects or boundaries in the subject of the image.

At S40, methods calculate an uncertainty metric to quantify the uncertainty provided for the previous prediction process. Each type of prediction will yield a distinct uncertainty metric. The uncertainty metric may be composed of multiple sub-metrics, for instance one or both aleatoric and epistemic uncertainties.

At S50, methods calculate a similarity metric to quantity the similarity between images in the training set (for example, the similarity between each pair of images or the similarity between each image and some standard image).

At S60, depending on the calculated similarity metric and the calculated uncertainty metric, methods select images from the unlabelled training set. These selected images are effectively added to the previous training examples, to form a new (or newly designed) training set for future iterations of the training process.

The selection of the designed training set is performed in a manner to lower the similarity between the selected images. The selection of the designed training set is performed in a manner to increase the uncertainty of the images forming the designed training set.

In contrast to existing methods, methods according to embodiments use similarity for images in combination with uncertainty metrics to select informative and diverse examples for active learning.

In addition, methods may optionally apply image perturbations to check and demonstrate improvements on safety and robustness of active learning strategies according to embodiments. For example, a similarity-based active learning for image classification under class imbalance (Zhang et al., 2018) was proposed, making use of a loss function in learning both feature representation and a similarity function jointly. Methods according to embodiments on the other hand build on already finished model architectures for segmentation and learn similarity from the data before adding the most representative and uncertain examples to the training set. The advantage is that models, which are already implemented and being used, may be adapted without change for use in an active learning setting and for follow-up tasks and fine-tuning on new datasets.

Relying on active learning, similarity metrics and Bayesian approaches to deep learning, methods according to an embodiment introduce a deep active learning framework for improving the annotation and the automatic image selection process for accurate and robust segmentation models. In this context, “deep” learning refers to neural networks with multiple layers; deep learning eliminates some of data pre-processing that is typically involved with conventional machine learning. These DL algorithms may ingest and process unstructured data, like text and images, and may automate feature extraction, removing at least some of the dependency on human experts.

An example framework according to embodiments utilise a state-of-the-art architecture with U-Net (Ronneberger et al., 2015) and EfficientNet (Mingxing and Quoc, 2019) backbone, and consists of modules for estimating uncertainty metrics with Bayesian deep learning, combining uncertainty and similarity metrics for better diversification of the training set for achieving better robustness.

The inventors have demonstrated that the example learning framework is appliable to, for example, optical coherence tomography (OCT) volumes for retinal layer segmentation. Taking advantage of Bayesian deep learning, active learning, and a similarity measure, it is possible to demonstrate the applicability of the example framework with OCT volume data for, for example, semantic segmentation. Other examples included later herein show the general applicability of the invention across all different image types. The network introduced above and detailed throughout may be used for all the different examples.

A commonly used metric in the field for quantifying the similarities between sample sets is the Jaccard index, or the intersection over union (IoU). Using a state-of-the-art model according to an embodiment, methods are able to achieve ˜0.8 IoU with only 112 labelled images without relying on unlabelled data. In comparison, 244 labelled images are needed to achieve 0.79 IoU using random sampling-requiring an expert to label more than twice as many images to achieve almost the same accuracy. Methods are capable of achieving ˜0.8 IoU with 148 labelled images using epistemic uncertainty only (that is, in the absence of aleatoric uncertainty and similarity). Using epistemic uncertainty only, one may cut the number of annotations needed by 39% and by 54% using epistemic uncertainty and a similarity metric.

Examples herein concentrate on medical images for the purpose of medical image segmentation, which need a model for semantic segmentation and a method to represent prediction uncertainties on image data. Of course, training methods according to embodiments are also applicable to other fields. Existing approaches such as RelayNet (Guha Roy et al., 2017) rely on architectures similar to a U-Net for retinal layer segmentation. In contrast, methods according to embodiments may alternatively employ a state-of-the-art U-Net based network with an EfficientNet backbone (Yakubovskiy, 2019) for this task.

To perform active learning and query new examples using uncertainty and similarity, methods according to embodiments rely on Bayesian deep learning and activate dropout during inference. That is, methods omit units (hidden and/or visible) during the training process of the DL model to reduce overfitting, preventing complex co-adaptations on training data. Of course, methods may additionally or alternatively activate dilution instead of (or in addition to) dropout. The uncertainty information in an example may be estimated with multiple stochastic forward passes (referred as Monte Carlo dropout). Example uncertainty estimation modules output two types of uncertainty: epistemic uncertainty and aleatoric uncertainty. Both types of uncertainties may be explored separately and in combination with each other and together with a similarity metric to compose a training data acquisition function.

Methods according to embodiments use different uncertainty metrics and the similarity measure for selecting additional examples. For example, in tasks involving semantic segmentation (that is, generally, labelling each pixel in images with a corresponding class, denoting what is being represented) for medical volumes or images, it is often important to query uncertain yet diverse examples. When one relies on uncertainty only, one might end up with similar looking images and thus images with similar scores for neighbouring images of a 3D medical volume. The inventors have come to realisation that combining uncertainty and a similarity measure gives a better acquisition strategy.

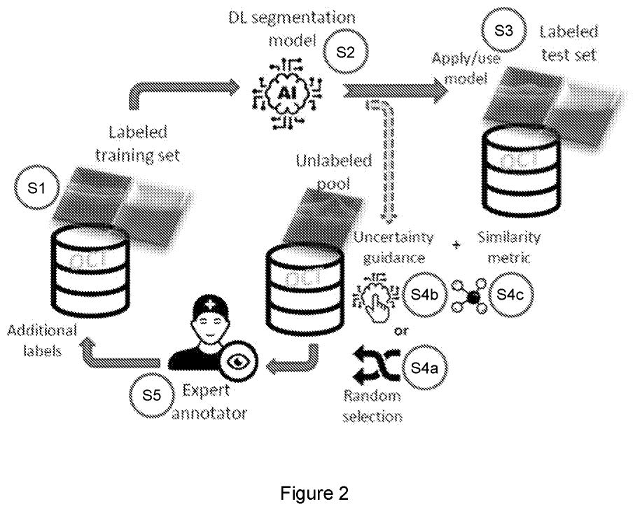

FIG. 2 demonstrates a method according to an embodiment. In the example implementation of the method according to embodiments, the method may start with a relatively small number of optical coherence tomography (OCT) scans (for example, four) and add new samples to the training set by ranking uncertainty information. Methods may select samples with high uncertainty, add images to the training set, and train a new model. The new model may be again used to calculate the uncertainty and start a new active learning iteration. In addition to uncertainty, methods employ a similarity metric to distinguish between images and better capture a diverse set of new samples, which are added to the training set.

At S1, the example method starts with a small quantity of labelled OCT training samples. At S2, the method trains a segmentation model using the labelled training set. At S3, methods checkpoint (acquire the weights of) the model using validation and apply the (trained) model on a labelled test set. For comparative purposes, methods at S4a may select new image samples randomly. At S4b and S4c, methods may select new image samples using estimated uncertainty metrics and similarity metrics. Finally, at S5, methods may present the new samples to the expert annotator (oracle). The newly (expertly) labelled samples may then be input as an accompaniment to the original OCT training sample set for iteratively improved results, by retraining and acquiring new, improved segmentation models.

To exemplify methods, the inventors use an OCT retinal layer dataset consisting of AMD (Age-related macular degeneration), DME (Diabetic macular edema) and normal/healthy volume scans.

The dataset is acquired from Rasti et al. (Macular OCT Classification Using a Multi-Scale Convolutional Neural Network Ensemble. IEEE Trans Med Imaging, 2018. 37 (4): pp. 1024-1034). The unlabelled dataset contains 148 OCT volumes split evenly between intermediate and late age related macular degeneration (AMD), Diabetic Macular Edema (DME) and Normal. The volumes are collected using the Heidelberg Spectralis platform. The dataset was collected using high speed settings, with a mix of scan densities between 20 and 61 B scans. From this larger dataset, approximately 20 volumes of mixed scan density were selected from each category. Images were arbitrarily assessed as being of good and of bad quality, with only good quality images selected for segmentation.

All images (739 AMD, 403 DME and 526 Normal) were segmented manually for the presence and location of the ILM, INL/OPL boundary, top of the IS/OS, the inner (IBRPE) and the outer boundary of the RPE (OBPRE). If layers disappear due to degeneration, graders were instructed to collapse a boundary to the layer below. Graders submitted completed (segmented) images, which were then compared between graders. Any disagreement greater than Intersection Over Union (IoU) 0.7 were returned to the graders for further review.

Disease, presence of fluid, and geographical atrophy were labelled at the Bscan level for all graded images. An additional 350 images were additionally graded for presence of four drusen subtypes, with bounding box labels using MakeSense.ai (an online tool for labelling images). The labelling suite is a custom development of the Hitachi Semantic Image Labeller https://github.com/hitachi-Automotive-And-Industry-Lab/semantic-segmentation-editor. Using a U-NET based and ReLayNet based segmentation all images in the dataset (148 volumes) were segmented.

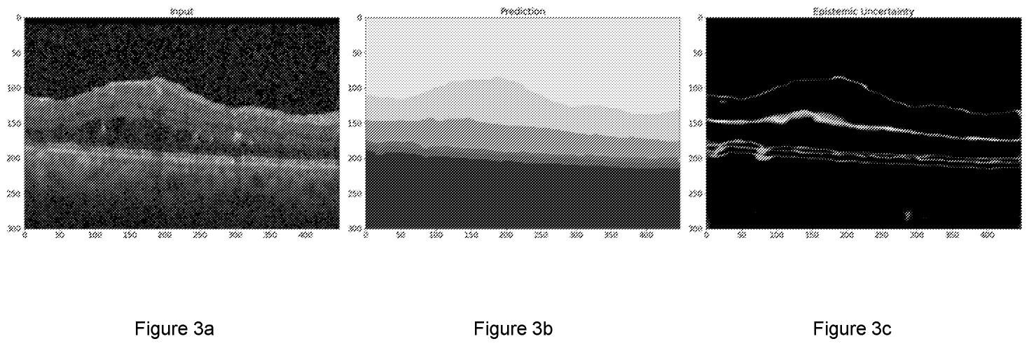

FIG. 3a illustrates an example OCT scan slice (image) from this dataset. Prior to processing, the inventors split the pool of OCT volumes representing different patients (1668 scans in total) into 20% training, 20% validation and 60% testing. The inventors opted for a relatively large test set ratio to ensure a more accurate calculation of model performance.

As an example implementation method of active learning for segmentation in digital images, the inventors have used a U-Net based convolutional neural network model with an EfficientNet backbone (feature extraction section) to train the baseline model. U-Net architectures contains two paths: a first path is the contraction path (encoder), used to capture the context in the image. The encoder may be considered as a traditional stack of convolutional and max pooling layers. The second path is a symmetric expanding path (decoder), used to enable precise localization using transposed convolutions. Thus, U-Net models are end-to-end fully convolutional network (FCN), i.e., they only contain Convolutional layers and do not contain any Dense layer. In this way, U-Net models may accept samples (images) of any size. EfficientNet is a convolutional neural network architecture and scaling method that uniformly scales all dimensions of depth/width/resolution using a compound coefficient. Unlike conventional architectures that arbitrary scale these factors, the EfficientNet scaling method uniformly scales network width, depth, and resolution with a set of fixed scaling coefficients. Of course, any other convolutional model architectures are suitable, so long as they permit acquisition of both uncertainty and similarity

Example model architectures are implemented using TensorFlow Keras. Of course, any other means of implementation of convolutional neural network architectures are suitable, such as Caffe or PyTorch.

FIG. 3b shows the prediction result of five retinal layers in the input scan as shown in FIG. 3a. The annotations (labels) on the OCT volume slices (images) include five boundaries and six different bands or classes to distinguish. The inventors use 512×512 pixel images for the input. The loss function in use is based on categorical cross-entropy and Jaccard loss; of course, other loss functions are also suitable. The evaluation metric in use is intersection over union (IoU). The network is trained with a starting learning rate of 0.001 using the Adam optimizer—a particular stochastic gradient descent method that is based on adaptive estimation of first-order and second-order moments; again, of course, other optimizers such as SGD or RMSprop may also be suitable. The learning is adjusted (with the function ReduceLROnPlateau) by reducing the learning rate when the validation metric stops improving with a patience of 15 epochs (that is, the number of epochs to wait before early stop if no progress is observed on the validation set). Additionally, the inventors apply early stopping with a patience of 50 after the first active learning iteration. The maximum number of epochs is set to 500. Of course, the skilled reader will appreciate other values for these parameters may be suitable.

### Uncertainty

As noted previously, the design of the uncertainty module within the larger neural network architecture is inspired by the MC dropout method. The inventors follow (Gal et al., 2016, Kendall et al., 2016) to estimate the epistemic uncertainty and (Kwon et al., 2020) to estimate the aleatoric uncertainty. To estimate the uncertainty with the segmentation model, one is interested in finding the posterior distribution over the convolutional weights, W, given the observed training data X and labels Y.

$$p ( W \vert X , Y ) \qquad ( Equation 1 )$$

The posterior distribution over the space of parameters by invoking Bayes' theorem is defined by:

$$p ( W \vert X , Y ) = \frac{p ( Y \vert X , W ) p ( W )}{p ( Y \vert X )} \qquad ( Equation 2 )$$

This distribution captures the most probable function parameters given the observed data. The predictive distribution may be defined as:

$$p ( {y}^{*} \vert {x}^{*} , X , Y ) = \int p ( {y}^{*} \vert {x}^{*} , W ) p ( W \vert X , Y ) dW \qquad ( Equation 3 )$$

for a new input x* and an output y*. The aleatoric uncertainty is estimated using the following formula where the first additive term denotes to epistemic (illustrated in FIG. 3c) and the second one to aleatoric uncertainty:

$$[ {y}^{*} \vert {x}^{*} ] = \frac{1}{T} {\sum }_{t = 1}^{T} ( - \overline{y} ) {( - \overline{y} )}^{T} + \frac{1}{T} {\sum }_{t = 1}^{T} ( diag ( ) - ( ) {( )}^{T} ) , \qquad ( Equation 4 )$$

where y̅=Σₜ₌₁^{T} [symbol] /T and [symbol] =softmax ( [symbol] (x*)). This approach follows the method suggested by Kwon et al. (2020), which has the advantage that one does not have to change the model or customize the loss function.

FIG. 3c shows the estimated epistemic (or model) uncertainty for the prediction shown in FIG. 3b. The uncertainty is estimated for all six classes and the sum is calculated over the whole input dimension of 512×512 pixels and different classes. The result is a single scalar value, which is used for ranking the images according to high to low uncertainty. The images for subsequent iterations for active learning are selected by uncertainty together with similarity, as follows.

### Similarity Module

Focusing on uncertainty only for the acquisition strategy has the disadvantage of adding uncertain but similarly looking images. This is a challenge for, for example, 3D medical volumes or video datasets (or, more generally, any data where data has more than two dimensions—for instance, in the case of 3D volumes, spatial information or, in case of video data, temporal information). Often medical volumes may have up to hundreds of images or slices. Yet, annotating all slices or frames (for example in non-medical data) is contra productive, time-consuming, and not representative (in the sense that all slices from one volume is not representative of a diverse population). A similarity measure may help to group images within medical volumes or frames of a video and the unlabelled pool to make a diverse selection possible for a better representative training set. Methods according to embodiments may implement a similarity module within the larger neural network architecture, configured to use a structural similarity index (such as Wang et al., 2004) to calculate a similarity value between every image. In case of N images, this results in a matrix with N×N entries. After clustering the images into different groups, one may change the selection strategy, where one ranks uncertainty and adds only single scans from different groups in a certain active learning iteration.

The structural similarity index (SSIM) is calculated as following: for the pixel location i=(i₁, i₂) in the set of coordinates Z², the local neighborhood pixels with radius r and c={j ∈Z², ∥i−j∥₁≤r}, the SSIM is defined for two spatial locations x and y from two images as:

$$SSIM ( x , y ) = ( \frac{2 \overline{x} \overline{y} + {c}_{1}}{{\overline{x}}^{2} + {\overline{y}}^{2} + {c}_{1}} ) ( \frac{2 {s}_{x , y} + {c}_{2}}{{s}_{x}^{2} + {s}_{y}^{2} + {c}_{2}} ) \qquad ( Equation 5 )$$

Two images are similar if the SSIM value is close to 1.0. The luminance of each signal may be estimated using the mean intensity as x and y. The standard deviations sₓ² and s_{y}² may be used to estimate the contrast. The signal may be normalized by the standard deviation and the structure term may be given by

$${s}_{x , y} = {\sum }_{j = 1}^{n} {w}_{i} ( {x}_{j} - \overline{x} ) ( {y}_{j} - \overline{y} ) , \qquad ( Equation 6 )$$

with the weights {wⱼ}ⱼ₌₁ⁿ such that Σⱼ₌₁ⁿwⱼ=1.

The SSIM measure may be used to group the individual images into clusters. During the selection process, methods may adjust the acquisition function by selecting high uncertainty samples from the same cluster only once during the same active learning iteration. This helps to prevent adding uncertain but similar looking images, and thus helps to select more diverse samples in the same iteration.

### Comparative Example

To determine the effectiveness of methods according to embodiments, the inventors have performed experiments comparing retinal layer segmentation using different acquisition functions. The baseline experiment is done with random selection (recall S4a in FIG. 2). In each case, methods start training with four images and add 4, 8, 12 and 24 images in subsequent iterations. The number of active learning iterations is 20. The numbers of samples or images for the iterations are 8, 12, 16, 20, 24, 32, 40, 48, 56, 64, 76, 88, 100, 112, 124, 148, 172, 196, 220 and 244 respectively. The following strategies are compared:
a) select images randomly,
b) select images based on high epistemic uncertainty,
c) select images based on high aleatoric uncertainty,
d) select images based on high epistemic uncertainty and similarity of the input images by clustering similar looking images
e) select images based on high aleatoric uncertainty and similarity, and
f) by combining epistemic, aleatoric uncertainty and similarity for selection.

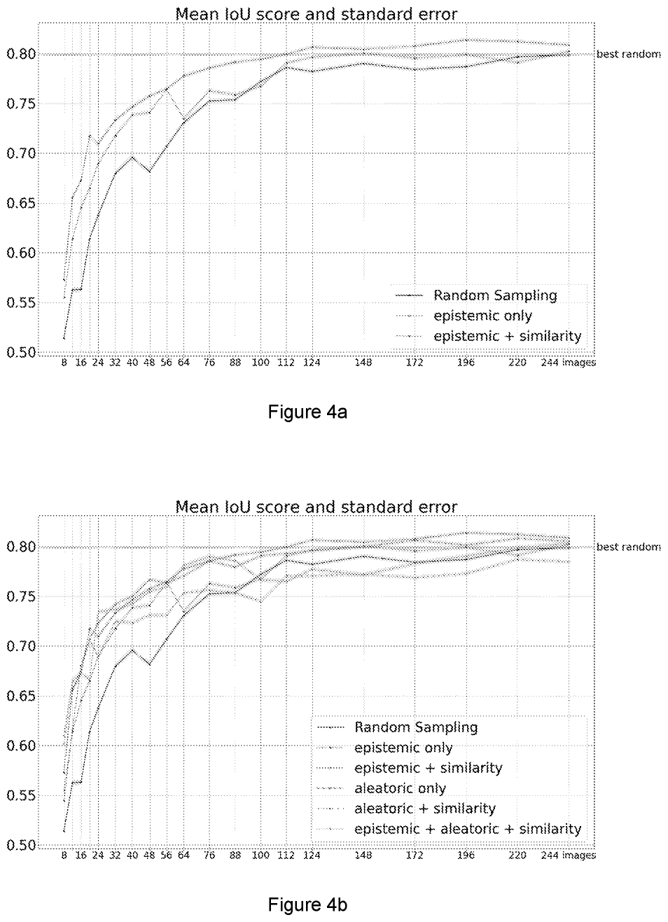

FIG. 4a illustrates the averaged results (with standard errors) of the IoU score for strategies a, b, and d after repeating the active learning procedure 10 times. Each of the 10 procedures uses different seeds for shuffling the training pool at the beginning of the individual run. In this way, the experiments account for the variation due to stratified sampling, especially at smaller data set sizes. As evidenced, including epistemic uncertainty for the purpose of image selection increases average IoU score beyond that of randomly selecting images in almost all image sample set sizes. Further, combining both epistemic uncertainty and similarity yields even greater IoU values for all image sample set sizes. For instance, for a sample set of 244 images, random sampling yields an IoU of just under 0.80; for the same sample set size, both epistemic only and epistemic in combination with similarity selection give IoU values above 0.80.

FIG. 4b illustrates the averaged results of the IoU score for all strategies (including strategies a, b and d as shows in FIG. 4a). Again, especially for sample sets up to 76 images in this instance, random image selection performs consistently worse (in terms of IoU value) than both epistemic and aleatoric uncertainty, alone and in combination (and alone and together in combination with similarity).

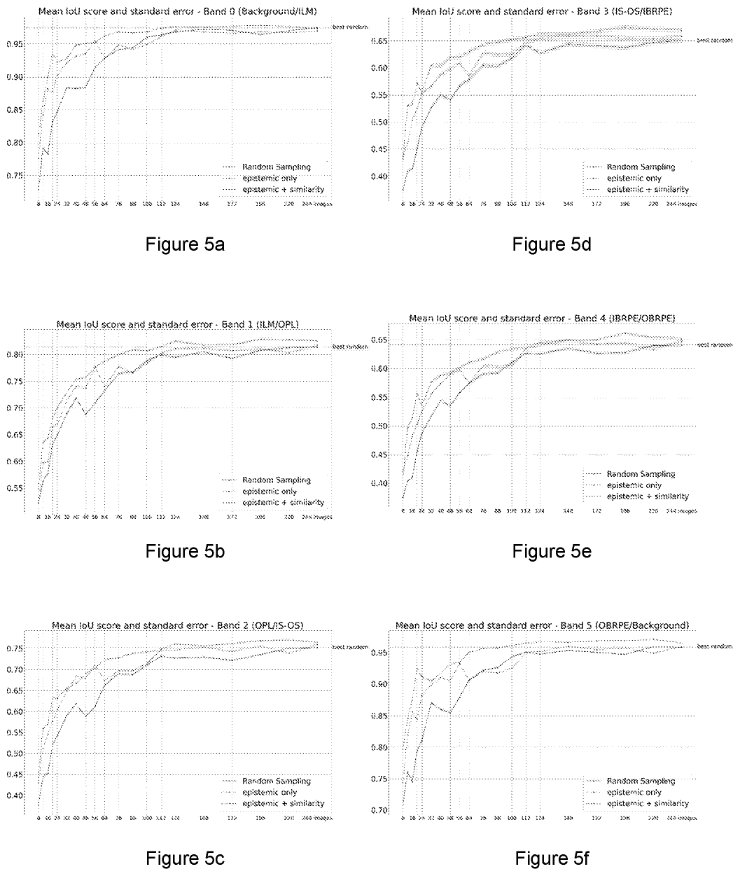

FIGS. 5a to 5f show results for the learning curve (development of IoU score with successive iterations, increasing image sample set size) for all individual retinal layers (bands 0 to 5) for strategies a, b, and d. Averaging of these individual retinal layer results are used in the calculation of the data used in FIGS. 4a and 4b.

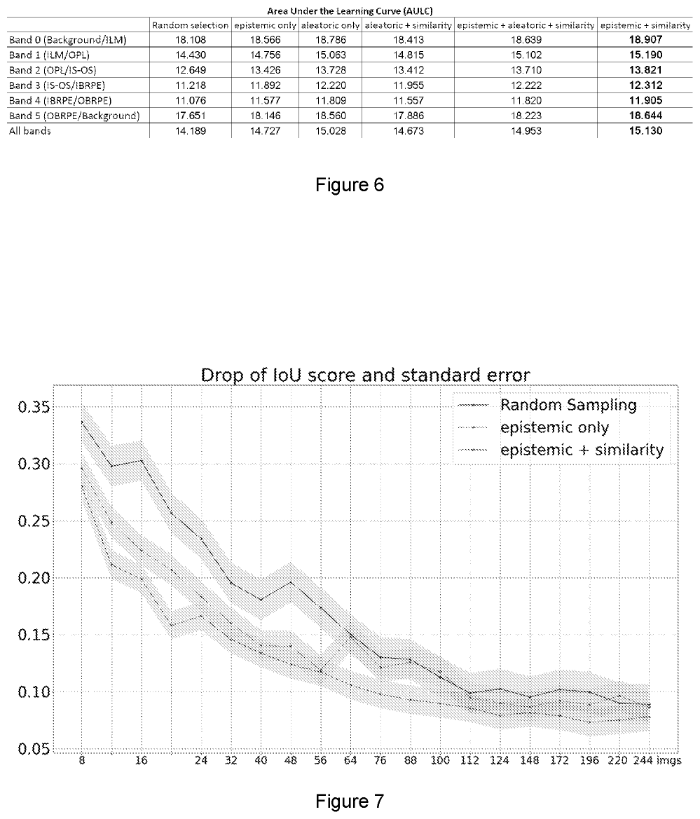

FIG. 6 is a table reporting the Area Under the Learning Curve (AULC) for all individual layer/band results and also for averaged results for IoU as a performance evaluation metric, for all strategies. AULC provides an aggregate measure of performance across all possible segregation/classification thresholds. One way of interpreting AULC is as the probability that the model ranks a random positive example more highly than a random negative example. As shown, combining uncertainty and similarity gives selection strategies that perform better than a random selection strategy.

Further testing of the models is performed on the test set and test sets with perturbations using gamma adjustment for changing brightness and contrast, and also on Gaussian noise perturbed test sets.

FIG. 7 shows (with standard errors) the drop of IoU score (test accuracy) when extending the test set with perturbed examples using Gaussian noise and gamma adjustment for contrast and brightness change. As with FIGS. 4a and 4b, this example uses random selection, epistemic uncertainty only, and epistemic uncertainty combined with similarity (strategies a, b, and d). As evidenced, combining epistemic uncertainty and similarity sees the most pronounced initial drop in IoU values for all image sample set sizes, indicating the improved robustness of the methods according to embodiments. That is, while random sampling eventually (after many iterations) begins to converge to IoU values matching those of non-perturbed sets, using epistemic uncertainty adjusted selection and epistemic uncertainty in combination with similarity adjusted selection sees this reduction much quicker.

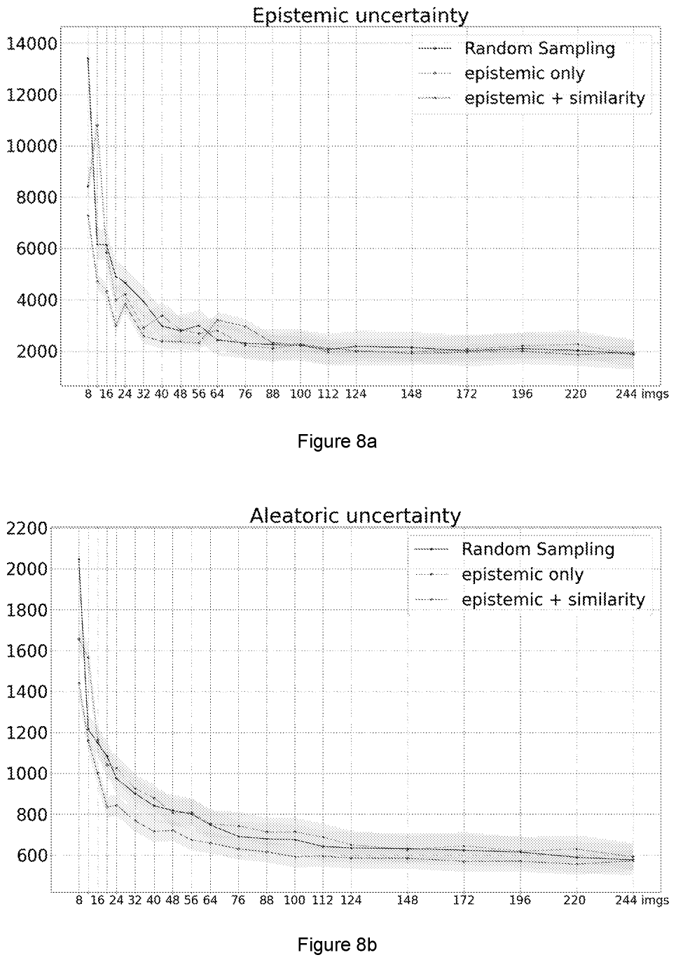

FIGS. 8a and 8b show the uncertainty contribution estimates for the active learning iterations together with standard error for epistemic and aleatoric uncertainty, respectively. The figures compare uncertainty contribution estimation values for epistemic and aleatoric uncertainty for random selection, epistemic uncertainty only, and a combination of epistemic uncertainty and similarity.

### Extension to Other Pathologies

Methods according to embodiments are not suited only to datasets comprising OCT retinal layers consisting of AMD (Age-related macular degeneration) and DME (Diabetic macular edema) but are applicable to other ophthalmology-related pathologies. Methods are also applicable to different datasets with a different disease by fine-tuning (for example, using the previously training weights and data as the initialization, selected according to best validation). That is, the DL segmentation models trained on the original extended test set containing AMD, DME and healthy scans are easily fine-tuned for applicability to other pathologies.

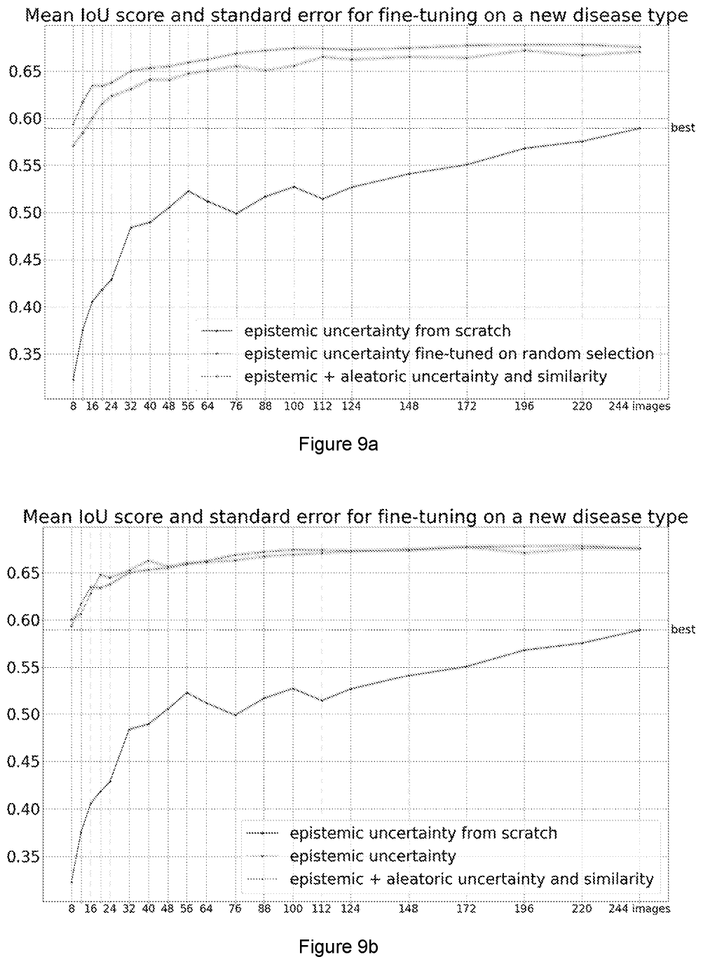

FIG. 9a shows results of experiments with retinal layer segmentation on OCT scans with Usher disease (where vision loss results from retinitis pigmentosa, a degeneration of the retinal cells).

The figure compares training a model from scratch vs. fine-tuning the model with epistemic uncertainty, using random selection vs. fine-tuning the model with epistemic uncertainty, aleatoric uncertainty, and similarity, using active learning in accordance with embodiments. The desire is to limit the number of required annotations for a new disease and associated dataset.

FIG. 9a visualizes the IoU performance on the three described training procedures. Using epistemic and aleatoric uncertainty in combination with similarity and fine-tuning on the best active learning model yields the highest mean IoU values for all image sample set sizes.

FIG. 9b compares training a model from scratch vs. fine-tuning the model with epistemic uncertainty, using active learning vs. fine-tuning the model with epistemic uncertainty, aleatoric uncertainty, and similarity, using active learning in accordance with embodiments.

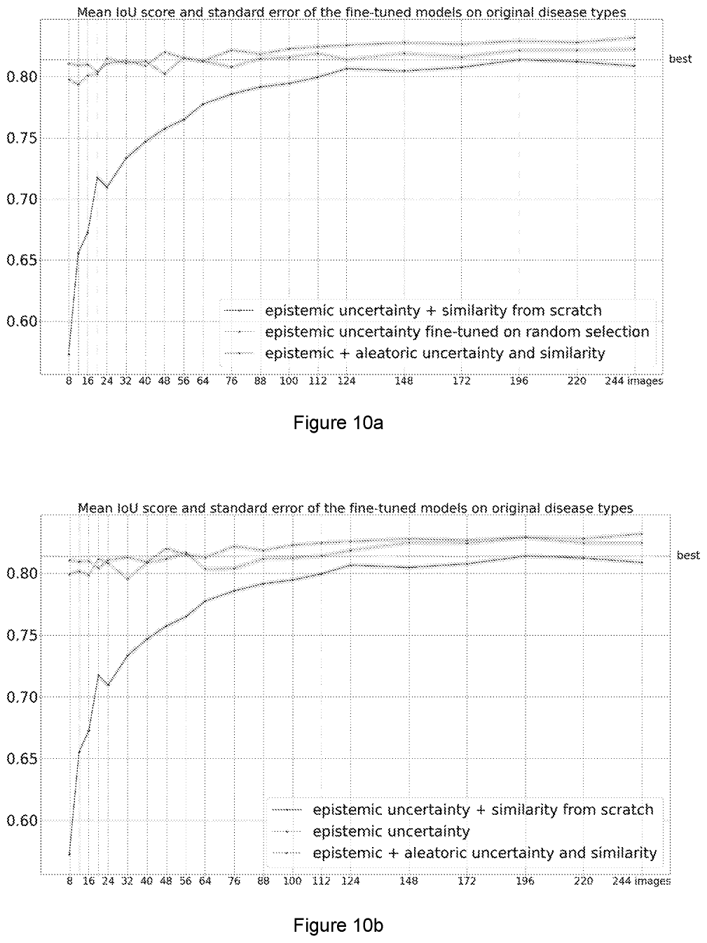

FIGS. 10a and 10b show the ability of the fine-tuned models to improve IoU values on the original extended test set containing AMD, DME and healthy scans, showcasing the performance gains. Note that, for comparison, the results of the active learning only (for epistemic in combination with similarity) are the same results as illustrated in FIG. 4a. By taking the original DL segmentation models (trained on AMD/DME), fine-tuning the models for Usher, then executing the models again on AMD/DME datasets yields consistently improved IoU values (for both a random selection and active-learning guided selection) for all image sample set sizes.

FIG. 10a, demonstrating fine-tuning the models for Usher syndrome and then executing the models again on AMD/DME/Normal datasets, compares epistemic uncertainty and similarity from scratch vs. epistemic uncertainty with fine-tuning on random selection vs. fine-tuning with epistemic uncertainty, aleatoric uncertainty, and similarity on active learning. FIG. 10b compares epistemic uncertainty and similarity from scratch vs. fine-tuning with epistemic uncertainty on active learning vs. fine-tuning with epistemic uncertainty, aleatoric uncertainty, and similarity on active learning.

### Synopsis

Medical image annotation is a time-intensive task, and one cannot estimate the number of needed examples to achieve a certain goal for accuracy. Therefore, active learning may help to reduce efforts to achieve set goals for accuracy. A comparison of different acquisition strategies and combinations of uncertainty metrics with a similarity measure indicate that methods according to embodiments suitably solve issues with existing medical image annotation.

Further, methods according to embodiments demonstrate robustness under different adversarial perturbations.

Uncertainty may be improved by using training frameworks according to methods according to embodiments. Experimental results demonstrate how active learning may be both assistive in producing better results and less vulnerable to adversarial examples.

In addition to training segmentation models from scratch, methods are also suitable for fine-tuning, to address new (previously unconsidered) diseases or pathologies; such fine-tunings show improvements for image vision tasks on the new disease test sets and also on the original (prior to fine-tuning) test sets. Using methods according to embodiments, it is possible to cut the need for annotation by more than 50% for training a model from scratch. And by using fine-tuning, it is possible to improve this value much more. Methods according to embodiments not only significantly speed up the annotation process for a new disease dataset, but also improve the ability to segment (or execute other computer vision tasks) on the original dataset.

In all cases, there is an advantage in using uncertainty for the selection. This advantage gets bigger when using similarity to select diverse samples. This is both the case for training a model from scratch and fine-tuning the model on a new dataset and a new disease type.

Methods according to embodiments are able to reduce the annotation effort and produce better results with less than half of the fully annotated dataset for training new models or fine-tuning on new datasets.

This new framework for uncertainty-based medical image segmentation for Deep Bayesian Active Learning includes modules for epistemic and aleatoric uncertainty and a similarity measure based on structural similarity index. The most informative and uncertain scans may be selected for manual annotation to improve and speed up the annotation and labelling process, thus saving and speeding up development of machine learning models for experts analyzing large amounts of data. This new framework aids experts in understanding diseases much faster and developing therapies much quicker. In addition, the uncertainty estimation and the active learning help improve human-Al collaboration for experts.

The skilled reader will appreciate that the approach using Bayesian deep learning for estimating uncertainty for unlabelled scans could further be modified to address or incorporate semi-supervised and self-supervised learning.

Further, the framework may also be extended to other image vision tasks, such as classification and detection tasks. That is, the active learning framework herein is agnostic to the architecture and task of a neural network and will help improve performance, learning efficiency, and feature robustness as long as uncertainty estimates can be made on a forward pass of the trained network.

For each prediction output, one may get a corresponding uncertainty from which one can derive a score for image selection. For example:

| Task | Prediction | Uncertainty metric |
|---|---|---|
| Classification | Image-level prediction of class. N | Sum of uncertainties from all N |
| | class probabilities. | classes for an image. |
| Segmentation | Pixel-level class prediction. | Sum of all H × W × N uncertainties |
| | H × W × N predictions. Where H is | for an image. |
| | the height (in pixels), W is the width | |
| | (in pixels), and N is the number of | |
| | classes. | |
| Object | 2 output predictions: (i) Regression | For uncertainty to make sense here, |
| detection | to get prediction of the bounding | we have to first group the predicted |
| | boxes (ii) classification of the | boxes from the multiple forward |
| | bounding boxes. | passes based on their overlap. Then |
| | | get the (i) uncertainty on the |
| | | bounding boxes of each bounding |
| | | box group identified, and (ii) |
| | | uncertainty on the N class |
| | | probability prediction for each |
| | | bounding box group identified. |
| | | The uncertainty metric is then the |
| | | sum of the bounding box uncertainty |
| | | and the bounding box class |
| | | uncertainty. |

### Extension to Other Fields

Methods according to embodiments are not limited solely to the medical field. The same techniques as disclosed herein also demonstrate success for non-medical datasets, such as CamVid (the Cambridge-driving Labelled Video Database). This database is a collection of videos with object class semantic labels, complete with metadata. The database provides ground truth labels that associate each pixel with one of 32 semantic classes.

The data was captured from the perspective of a driving automobile. The driving scenario increases the number and heterogeneity of the observed object classes. Over ten minutes of 30 Hz video footage is included in the dataset, with corresponding semantically labelled images at 1 Hz and in part, 15 Hz.

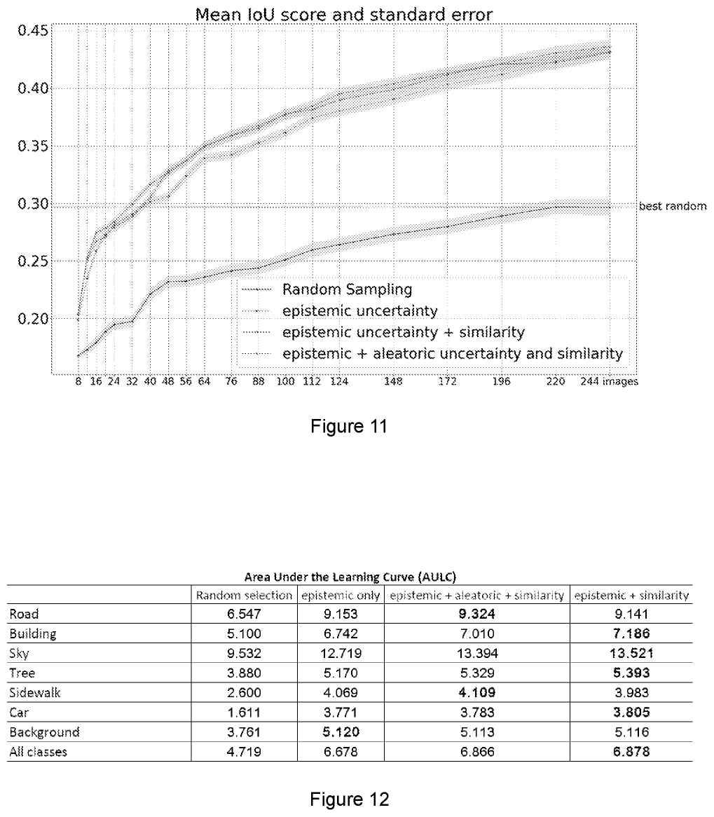

FIG. 11 demonstrates the IoU learning curve for methods applied to the CamVid dataset. As with the medical-imaging dataset, numerous strategies are compared (strategies a, b, d, and f, as introduced above). That is, the figure demonstrates: selecting images randomly; selecting images based on high epistemic uncertainty; selecting images based on high epistemic uncertainty and similarity of the input images by clustering similar looking images; and selecting images by combining epistemic, aleatoric uncertainty and similarity. The figure presents mean IoU scores and standard errors for all classes of objects in images of the dataset.

FIG. 12 is a table of AULC values for strategies a, b, d, and f, quantitatively demonstrating the success of methods according to embodiments.

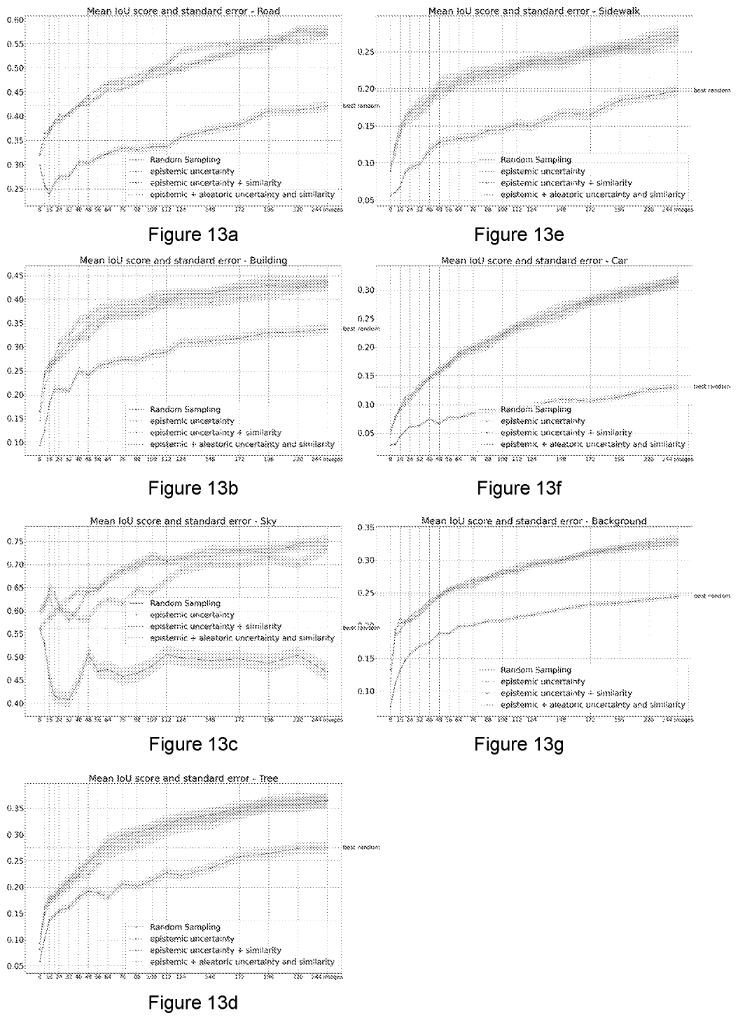

FIGS. 13a to 13g demonstrate the IoU learning curves for each of the individual classes considered in this trial: road, building, sky, tree, sidewalk, car, and background, respectively. The AULC results for each individual class are included in the table in FIG. 12.

As evidenced, including epistemic uncertainty for the purpose of image selection increases average IoU score beyond that of randomly selecting images in almost all image sample set sizes. Further, both the combination of epistemic uncertainty with similarity and the combination with epistemic and aleatoric uncertainty with similarity routinely yields even greater IoU values for all image sample set sizes. The inventors have thus demonstrated the applicability of methods according to embodiments in a wide range of fields in image vision.

### Pre-Processing for Flattening and Alignment

When using the SSIM as the similarity measure of choice (or indeed, when using any other suitable similarity measure), optionally, a pre-processing step may be performed to flatten and align images prior to processing through the system. For instance, in the case of OCT retinal scan slices, the OBRPE layer between the Bscans may be aligned. In this way, efficiency of the subsequent processing steps may be improved. This pre-processing step may be performed to remove or reduce the effect of any spatial translation of the retinal layers between the images from biasing the similarity measure. That is, during sample selection, the training set may be built from the unlabelled training pool and the unlabelled set/images may be pre-processed and prepared for similarity calculation. Pre-processing may be performed on any dataset prior to training the neural network, and additionally or alternatively may be performed on any dataset for use with the trained neural network.

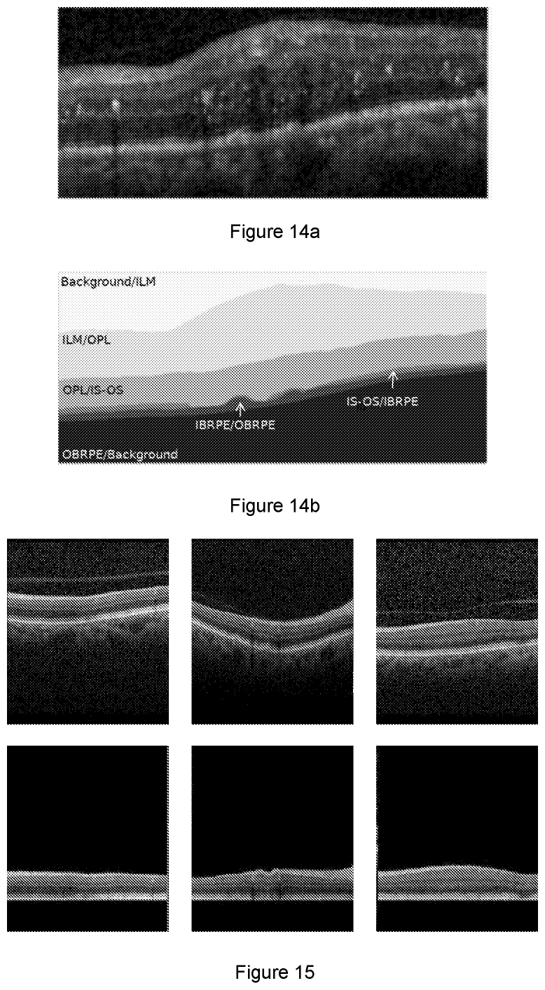

FIG. 14a is an example retinal OCT scan slice. FIG. 14b is a ground truth semantic segmentation or retinal layer segmentation (e.g., to delineate retinal layers within an OCT slice by assigning labels to every pixel in the scan) of the same example retinal OCT scan slice. The segmentation indicates the retinal boundary layer names, as discussed above.

FIG. 15 is a set of three pairs of retinal OCT scan slices. Each of the original images (top row) are pre-processed in order to flatten the OBRPE layer in the individual scan and to align the OBRPE layers of all scans; note how the interface between the OBRPE and the IBRPE in each pre-processed image (bottom row) is at the same vertical position. In the present case, pre-processing is performed using the technique described by Zhou, H. et al. The skilled reader will appreciate that any other suitable image pre-processing techniques may be used to provide some uniformity to images prior to any prediction task.

### Demonstrating Robustness

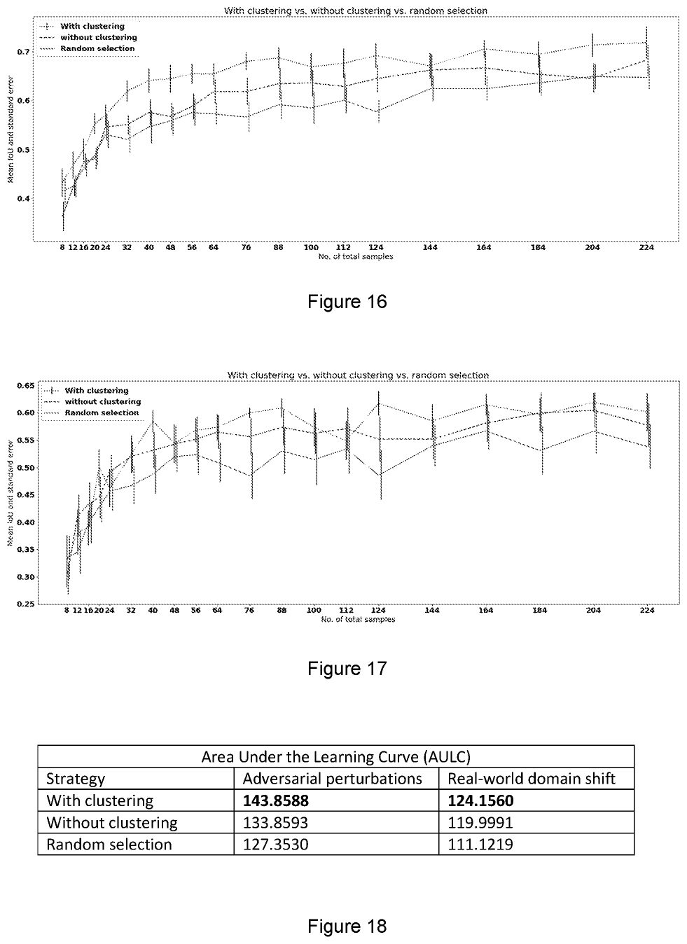

As discussed throughout, embodiments are well suited for improving performance robustness relative to existing techniques. FIG. 16 is a graph demonstrating the robustness of methods according to embodiments when undergoing adversarial perturbations. In this case, scans (images) acquired using the Heidelberg Spectralis platform (the Rasti et al. dataset, as described above) undergo two image perturbations: the addition of Gaussian noise; and the change of brightness and contrast, by Gamma adjustment. FIG. 16 provides a comparison of IoU for a varying number of perturbed images (from datasets of 8 to 224 images), when these perturbed images are passed through a trained neural network.

The top-most curve (that is, top-most for all sample sizes; doted line) is the IoU where the perturbed images are selected according to embodiments (namely, according to clustering by similarity, followed by selection—from amongst the clusters—by uncertainty). The middle curve (that is, middle for most sample sizes, in particular the middle curve at a sample size of 88 perturbed images; dashed line) is the IoU where the perturbed images are selected without any clustering by similarity, and are, instead, selected by uncertainty and similarity. The bottom-most curve (that is, bottom-most for most sample sizes, in particular the bottom-most curve at a sample size of 88 perturbed images; solid line) is the IoU where the perturbed images are selected randomly. For all sample sizes, clustering of perturbed images by similarity followed by selection by uncertainty, when passed through a pre-trained neural network, yields the highest IoU values, indicating excellent performance when the trained neural network is faced with a new (albeit similar in this case) dataset. Incidentally, a Bayesian Signed-Rank Test was performed for statistical significance.

FIG. 17 is a graph demonstrating the robustness of methods according to embodiments when faced with real-world shifts. Here, the pre-trained neural network model is used to process (segment) new datasets in the form of AMD scans, acquired using a different scanning device. In this case, AMD scans are acquired using a Bioptigen OCT scanning system—that is, the AMD scans are acquired using a different scanning device, provided by a different vendor to that used for acquisition of the Rasti et al. dataset, described above (Heidelberg). FIG. 17 provides a comparison of IoU for a varying number of real-world shifted images (from datasets of 8 to 224 images), when these real-world shifted images are passed through a trained neural network.

The top-most curve (that is, top-most for most sample sizes, in particular the top-most curve at a sample size of 124 images; dotted line) is the IoU where the real-world shifted images are selected according to embodiments (namely, according to clustering by similarity, followed by selection—from amongst the clusters—by uncertainty). The middle curve (that is, middle for most sample sizes, in particular the middle curve at a sample size of 124 images; dashed line) is the IoU where the real-world shifted images are selected without any clustering by similarity, and are, instead, selected by uncertainty and similarity. The bottom-most curve (that is, bottom-most for most sample sizes, in particular the bottom-most curve at a sample size of 124 images; solid line) is the IoU where the real-world shifted images are selected randomly. For all sample sizes, clustering of real-world shifted images by similarity followed by selection by uncertainty, when passed through a pre-trained neural network, yields the highest IoU values, indicating excellent performance when the trained neural network is faced with a new dataset, acquired from a different source than that of the source used for training images. Thus, embodiments are robust to changes to unseen devices, different devices, and/or different data sources. Again, a Bayesian Signed-Rank Test was performed for statistical significance.

Notably, for both adversarial (or synthetic) perturbations and real-world shift, the use of uncertainty and similarity (with and without clustering) routinely perform better than random selection of images. This is particularly evident at small sample sizes (e.g., from samples of 8 to 40 in FIGS. 16 and 17), which demonstrates the efficacy of techniques disclosed herein to smaller datasets, which are commonplace in fields such as medical imaging.

FIG. 18 is a table of AULC values for both adversarial perturbations and real-world domain shifts, which quantitatively demonstrates the effectiveness of methods according to embodiments (with and without clustering) across the entire breadth of the sample sizes. For the adversarial perturbation case, clustering yields an AULC of 143.9, without selection yields 133.86, and random selection yields only 127.4. For the real-world domain shift case, clustering yields an AULC of 124.2, without selection yields 120.0, and random selection yields only 111.1.

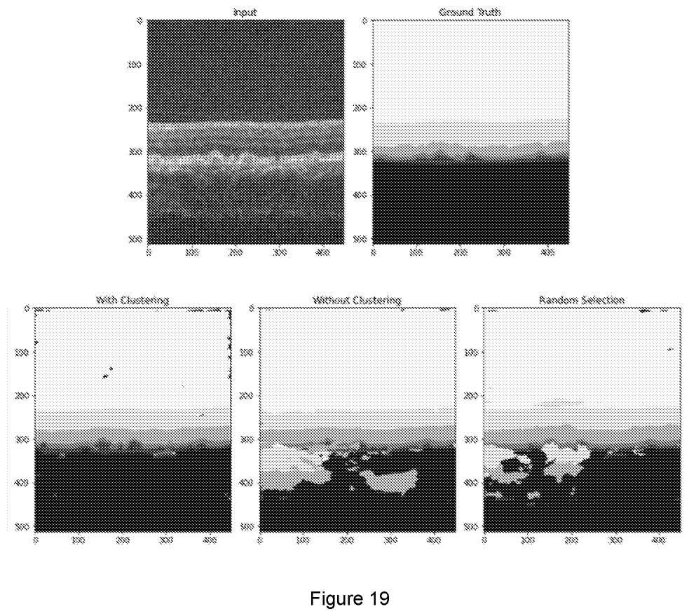

FIG. 19 provides a qualitative indication of segmentation on a single image, for the real-world domain shift case. That is, a single image (input) acquired using the Bioptigen OCT scanning system is passed through a neural network trained using images acquired with a Heidelberg system, as described above. The frame headed “Ground Truth” demonstrates the manually segmented boundary layers. Immediately, one observes the improved accuracy of the “with clustering” approach relative to, in particular, the random selection approach.

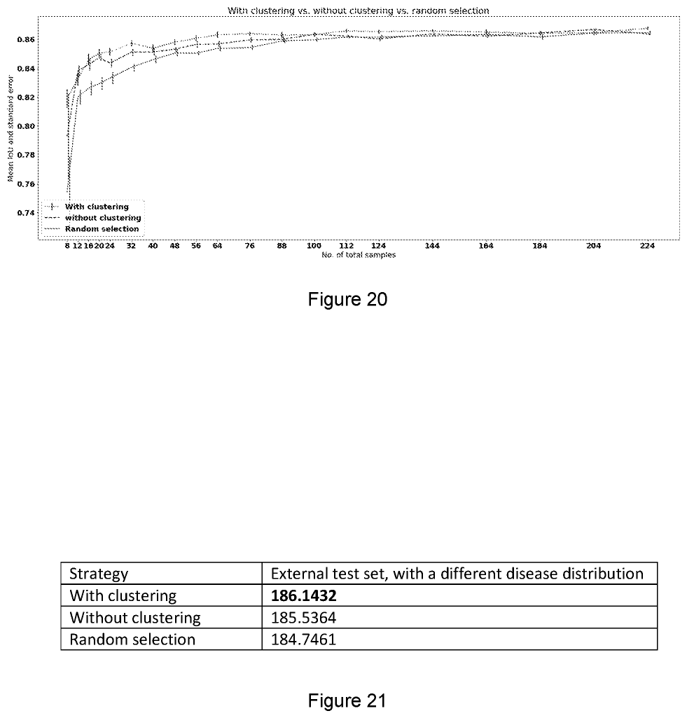

In addition to improved robustness in the event of image perturbations and in the event of real-world shift due to different data sources, methods according to embodiments when applied to medical images are also robust to changes in underlying diseases within scans. FIG. 20 demonstrates a real-world shift in the sense of that the neural network is trained using images acquired using the Heidelberg system (detailed above) and segmentation is performed using the trained neural network and using new images where DME is present. FIG. 20 provides a comparison of IoU for a varying number of real-world different disease images, where a different disease distribution is present (from datasets of 8 to 224 images, containing only DME scans), when these real-world different disease images are passed through a trained neural network. The DME-only dataset is adapted from the work of Chiu, J. S. et al.

The top-most curve (that is, top-most for most sample sizes, in particular the top-most curve at a sample size of 32 images; dotted line) is the IoU where the real-world different disease images are selected according to embodiments (namely, according to clustering by similarity, followed by selection—from amongst the clusters—by uncertainty). The middle curve (that is, middle for most sample sizes, in particular the middle curve at a sample size of 32 images; dashed line) is the IoU where the real-world different disease images are selected without any clustering by similarity, and are, instead, selected by uncertainty and similarity. The bottom-most curve (that is, bottom-most for most sample sizes, in particular the bottom-most curve at a sample size of 124 images; solid line) is the IoU where the real-world different disease images are selected randomly. For all sample sizes, clustering of real-world different disease images by similarity followed by selection by uncertainty, when passed through a pre-trained neural network, yields the highest IoU values, indicating excellent performance when the trained neural network is faced with a new (albeit similar) dataset. Thus, embodiments are robust to changes to unseen devices, different devices, and/or different data sources. Again, a Bayesian Signed-Rank Test was performed for statistical significance.

FIG. 21 is a table of AULC values for real-world different disease shifts, which quantitatively demonstrates the effectiveness of methods according to embodiments (with and without clustering) across the entire breadth of the sample sizes. Clustering yields an AULC of 186.1, without selection yields 185.5, and random selection yields only 184.7.

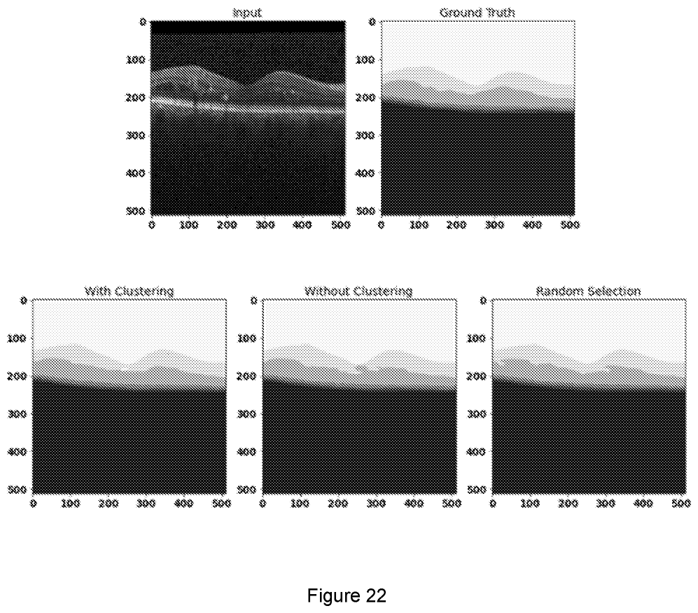

FIG. 22 provides a qualitative indication of segmentation on a single image, for the real-world different disease case. That is, a single image (input), in which DME is present, is passed through a neural network trained using the images acquired with a Heidelberg system, as described above. The frame headed “Ground Truth” demonstrates the manually segmented boundary layers. Again, immediately, one observes the improved accuracy of the “with clustering” approach relative to, in particular, the random selection approach.

The below table summarises the datasets used here to demonstrate robustness. Note that the Rasti et al. dataset is used—prior to any adversarial shift—for training the neural network, as discussed above.

| | | Demonstrating | | |
|---|---|---|---|---|
| Dataset | No. | robustness | | Acquisition |
| Name | Scans | under . . . | Diseases | Device |
| Rasti et al. | 651 | adversarial shift | AMD | Heidelberg |
| | | (see FIG. 16) | & DME | |
| | | | & Normal | |
| Bioptigen | 145 | domain shift | AMD | Bioptigen |
| Farsui et al. | | (see FIG. 17) | | |
| Duke/Chiu | 110 | shift in distribution, | DME | Heidelberg |
| Chui et al. | | population, hospital | | |
| | | (see FIG. 20) | | |

Both the Bioptigen (Farsui et al.) and the Duke/Shiu (Chiu et al.) datasets are used here for testing the model and not for training.

### Hardware

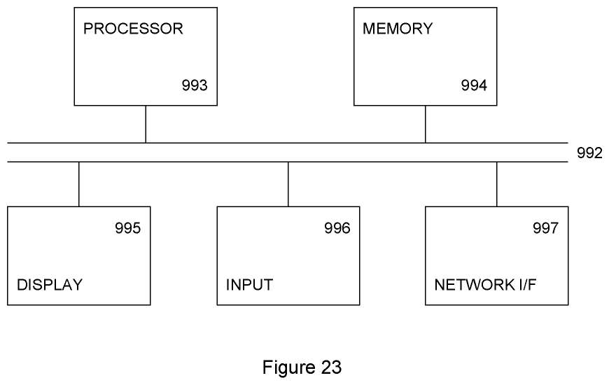

FIG. 23 is a block diagram of a computing device, such as a data storage server, which embodies the present invention and which may be used to implement aspects of the methods for active learning for computer vision tasks, as described herein. The computing device comprises a processor 993, and memory 994. Optionally, the computing device also includes a network interface 997 for communication with other computing devices.

For example, an embodiment may be composed of a network of such computing devices. Optionally, the computing device also includes one or more input mechanisms such as keyboard and mouse 996, and a display unit such as one or more monitors 995. The components are connectable to one another via a bus 992.

The memory 994 may include a computer readable medium, a term which may refer to a single medium or multiple media (e.g., a centralised or distributed database and/or associated caches and servers) configured to carry computer-executable instructions or have data structures stored thereon. Computer-executable instructions may include, for example, instructions and data accessible by and causing a general-purpose computer, special purpose computer, or special purpose processing device (e.g., one or more processors) to perform one or more functions or operations. Thus, the term “computer-readable storage medium” may also include any medium that is capable of storing, encoding, or carrying a set of instructions for execution by the machine and that cause the machine to perform any one or more of the methods of the present disclosure. The term “computer-readable storage medium” may accordingly be taken to include, but not be limited to, solid-state memories, optical media, and magnetic media. By way of example, and not limitation, such computer-readable media may include non-transitory computer-readable storage media, including Random Access Memory (RAM), Read-Only Memory (ROM), Electrically Erasable Programmable Read-Only Memory (EEPROM), Compact Disc Read-Only Memory (CD-ROM) or other optical disk storage, magnetic disk storage or other magnetic storage devices, flash memory devices (e.g., solid state memory devices).

The processor 993 is configured to control the computing device 400 and to execute processing operations, for example executing code stored in the memory 404 to implement the various different functions of the active learning method, as described here and in the claims.

The memory 994 may store data being read and written by the processor 993, for example data from training or segmentation tasks executing on the processor 993. As referred to herein, a processor 993 may include one or more general-purpose processing devices such as a microprocessor, central processing unit, GPU, or the like. The processor may include a complex instruction set computing (CISC) microprocessor, reduced instruction set computing (RISC) microprocessor, very long instruction word (VLIW) microprocessor, or a processor implementing other instruction sets or processors implementing a combination of instruction sets. The processor 993 may also include one or more special-purpose processing devices such as an application specific integrated circuit (ASIC), a field programmable gate array (FPGA), a digital signal processor (DSP), network processor, or the like. In one or more embodiments, a processor 993 is configured to execute instructions for performing the operations and steps discussed herein.

The network interface (network I/F) 997 may be connected to a network, such as the Internet, and is connectable to other computing devices via the network. The network I/F 997 may control data input/output from/to other apparatuses via the network.

Methods embodying aspects of the present invention may be carried out on a computing device such as that illustrated in FIG. 23. Such a computing device need not have every component illustrated in FIG. 23 and may be composed of a subset of those components. A method embodying aspects of the present invention may be carried out by a single computing device in communication with one or more data storage servers via a network or by a plurality of computing devices operating in cooperation with one another. Cloud services implementing computing devices may be deployed.

### REFERENCES

David A. Cohn, Zoubin Ghahramani, and Michael I. Jordan. 1996. Active learning with statistical models. J. Artif. Int. Res. 4, 1 (January 1996), 129-145.
Yang, Lin & Zhang, Yizhe & Chen, Jianxu & Zhang, Siyuan & Chen, Danny. (2017). Suggestive Annotation: A Deep Active Learning Framework for Biomedical Image Segmentation.
Yarin Gal and Zoubin Ghahramani. 2016. Dropout as a Bayesian approximation: representing model uncertainty in deep learning. In Proceedings of the 33rd International Conference on International Conference on Machine Learning-Volume 48 (ICML′16). JMLR.org, 1050-1059.
Kwon, Yongchan & Won, Joong-Ho & Kim, Beom & Paik, Myunghee. (2019). Uncertainty quantification using Bayesian neural networks in classification: Application to biomedical image segmentation. Computational Statistics & Data Analysis. 142. 106816. 10.1016/j.csda.2019.106816.
Zhang, Zhenxi & Li, Jie & Zhong, Zhusi & Jiao, Zhicheng & Gao, Xinbo. (2019). A sparse annotation strategy based on attention-guided active learning for 3D medical image segmentation.
Zhou, Zongwei & Shin, Jae & Zhang, Lei & Gurudu, Suryakanth & Gotway, Michael & Liang, Jianming. (2017). Fine-Tuning Convolutional Neural Networks for Biomedical Image Analysis: Actively and Incrementally. Proceedings. IEEE Computer Society Conference on Computer Vision and Pattern Recognition. 2017. 10.1109/CVPR.2017.506.
Gorriz Blanch, Marc and Carlier, Axel and Faure, Emmanuel and Giro I Nieto, Xavier. Cost-Effective Active Learning for Melanoma Segmentation. (2017) In: 31st Conference on Machine Learning for Health: Workshop at NIPS 2017 (ML4H 2017), 8 Dec. 2017-8 Dec. 2017 (Long Beach, California, United States).
Li, Bo and T. S. Alstrøm. “On uncertainty estimation in active learning for image segmentation.” ArXiv abs/2007.06364 (2020).
Gal, Y. et al. “Deep Bayesian Active Learning with Image Data.” ArXiv abs/1703.02910 (2017).
W. H. Beluch, T. Genewein, A. Nurnberger and J. M. Kohler, “The Power of Ensembles for Active Learning in Image Classification,” 2018 IEEE/CVF Conference on Computer Vision and Pattern Recognition, 2018, pp. 9368-9377, doi: 10.1109/CVPR.2018.00976.
Ronneberger, Olaf & Fischer, Philipp & Brox, Thomas. (2015). U-Net: Convolutional Networks for Biomedical Image Segmentation. LNCS. 9351. 234-241. 10.1007/978-3-319-24574-4_28.
Guha Roy, Abhijit & Conjeti, Sailesh & Karri, Sri Phani & Sheet, Debdoot & Katouzian, Amin & Wachinger, Christian & Navab, Nassir. (2017). ReLayNet: Retinal Layer and Fluid Segmentation of Macular Optical Coherence Tomography using Fully Convolutional Network. Biomedical Optics Express. 8. 10.1364/BOE.8.003627.
Rasti, R., et al., Macular OCT Classification Using a Multi-Scale Convolutional Neural Network Ensemble. IEEE Trans Med Imaging, 2018. 37 (4): p. 1024-1034.
CamVid: Brostow, Shotton, Fauqueur, Cipolla (2008). Segmentation and Recognition Using Structure from Motion Point Clouds, ECCV 2008.
Wang, Zhou & Bovik, Alan & Sheikh, Hamid. (2005). Structural Similarity Based Image Quality Assessment. Digital Video Image Quality and Perceptual Coding, Ser. Series in Signal Processing and Communications. 10.1201/9781420027822.ch7.
Zhou H, Dai Y, Gregori G, Rosenfeld P R, Duncan J L, Schwartz D M, Wang R K. Automated morphometric measurement of the retinal pigment epithelium complex and choriocapillaris using swept source OCT. Biomed Opt Express. 2020 Mar. 6;11 (4): 1834-1850. doi: 10.1364/BOE.385113. PMID: 32341851; PMCID: PMC7173887.
Chiu S J, Allingham M J, Mettu P S, Cousins S W, Izatt J A, Farsiu S. Kernel regression based segmentation of optical coherence tomography images with diabetic macular edema. Biomed Opt Express 2015; 6:1172-1194.
Farsiu, Sina & Chiu, Stephanie & O'Connell, Rachelle & Folgar, Francisco & Yuan, Eric & Izatt, Joseph & Toth, Cynthia. (2014). Quantitative Classification of Eyes with and without Intermediate Age-related Macular Degeneration Using Optical Coherence Tomography. Ophthalmology. 121. 162-172. 10.1016/j.ophtha.2013.07.01.3.

The invention claimed is:

1. A computer-implemented method of active learning for computer vision in digital images, comprising:
inputting labelled image training examples into an artificial neural network in a training phase;
training a neural network model using the labelled training examples;
carrying out a prediction task on each image of an unlabelled training set of unlabelled, unseen images using the model;
calculating an uncertainty metric for the predictions in each image of the unlabelled training set;
calculating a similarity metric for the unlabelled training set representing similarities between the images in the training set; and
selecting images from the unlabelled training set, in dependence upon both the similarity metric and the uncertainty metric of each image, to design a training set for labelling, which tends to both lower the similarity between the selected images and increase the uncertainty of the selected images.

2. The method according to claim 1, further comprising:
outputting the training set for labelling to an expert;
inputting the same images of the training set for labelling further including labels added by the expert as a labelled training set into the artificial neural network;
further training the model using the labelled training set; and
carrying out the prediction task on new images in an inference phase using the refined segmentation model.

3. The method according to claim 1, wherein the uncertainty metric is based on a Monte Carlo, MC, dropout method of estimating uncertainty.

4. The method according to claim 1, wherein the uncertainty metric considers epistemic uncertainty and/or aleatoric uncertainty.

5. The method according to claim 4, wherein the uncertainty metric is a global uncertainty metric incorporating estimation of both an epistemic uncertainty metric and an aleatoric uncertainty metric.

6. The method according to claim 5, wherein the global uncertainty metric is estimated and used for ranking of images.

7. The method according to claim 1, wherein the uncertainty metric uses Bayes' theorem to find a posterior distribution over convolutional weights W, given observed training data X and labels Y.

8. The method according to claim 1, wherein the prediction is segmentation, and the uncertainty is uncertainty of pixel classification.

9. The method according to claim 8, wherein the uncertainty of pixel classification is estimated and summed for each classification and over each pixel of the image to give a single scalar value.

10. The method according to claim 1, wherein the similarity algorithm groups the images by clustering of similar images.

11. The method according to claim 10, wherein the similarity algorithm clusters similar images into a single group, to provide a number of groups K each containing similar images and calculates a structural similarity index in matrix form of N×N entries giving similarity between every image in the unlabelled training set, where N×N is used to find the K clusters.

12. The method according to claim 11, wherein the image with the highest uncertainty level of the uncertainty metric in each group is selected for labelling from the training set.

13. The method according to claim 12, wherein in any subsequent active learning iteration, further selection takes the highest uncertainty level image from the remaining unselected images in each group.

14. The method according to claim 1, further comprising:
extending the labelled training examples by adding perturbed images of the labelled training examples.

15. The method according to claim 14, wherein the perturbed images of the labelled training examples include Gaussian noise and/or gamma adjustment to change contrast and brightness.

16. The method according to claim 1, wherein the images are medical images.

17. The method according to claim 16, wherein the medical images are volumetric data or videos.

18. The method according to claim 16, wherein the medical images are provided by scans or cameras.

19. A data processing apparatus comprising: a memory; and a processor, wherein the memory comprises instructions, and
the processor is configured to execute the instructions to:
accept input of labelled image training examples into an artificial neural network in a training phase;
train a neural network model using the labelled training examples;
carry out a prediction task on each image of an unlabelled training set of unlabelled, unseen images using the model;
calculate an uncertainty metric for the predictions in each image of the unlabelled training set;
calculate a similarity metric for the unlabelled training set representing similarities between the images in the training set; and
select images from the unlabelled training set, in dependence upon both the similarity metric and the uncertainty metric of each image, to design a training set for labelling, which tends to both lower the similarity between the selected images and increase the uncertainty of the selected images.

20. A non-transitory computer-readable medium comprising instructions, which, when the program is executed by a computer, cause the computer to:
accept input of labelled image training examples into an artificial neural network in a training phase;
train a neural network model using the labelled training examples;
carry out a prediction task on each image of an unlabelled training set of unlabelled, unseen images using the model;
calculate an uncertainty metric for the predictions in each image of the unlabelled training set;
calculate a similarity metric for the unlabelled training set representing similarities between the images in the training set; and
select images from the unlabelled training set, in dependence upon both the similarity metric and the uncertainty metric of each image, to design a training set for labelling, which tends to both lower the similarity between the selected images and increase the uncertainty of the selected images.
